(19) United States
(12) Patent Application Publication
(10) Pub. No.: US 2020/0108264 A1
(43) Pub. Date: Apr. 9, 2020

(54) METHOD TO IMPROVE OUTCOMES DURING NEGOTIATIONS

(71) Applicants: Joel Steven Goldberg, Hillsborough, NC (US); Craig L. Jackson, Hillsborough, NC (US)

(72) Inventors: Joel Steven Goldberg, Hillsborough, NC (US); Craig L. Jackson, Hillsborough, NC (US)

(21) Appl. No.: 16/706,760

(22) Filed: Dec. 8, 2019

(51) Int. Cl. A61N 2/00; G16H 20/30; G16H 20/70; A61B 5/0476; A61N 2/02; A61B 5/00

(52) U.S. Cl. CPC A61N 2/006; G16H 20/30; A61B 5/4836; A61B 5/0476; A61N 2/02; G16H 20/70

(57) ABSTRACT

Hyperscanning studies of the electrical activity of the brains of dyads and groups has shown that cooperative decision-making is associated with inter brain partial synchronization. Cooperative decision-making may be essential for long term resolution of conflict among nations. This invention demonstrates that synchronization of two Chua's circuits can be achieved through induction, and a single Chua's circuit can transition from chaotic to periodic activity with frequency dependent square wave inputs, models of action potentials. Extrapolating these in vitro findings suggests that conflict negotiations can be improved with electrophysiological monitoring of participants and with external electromagnetic energy targeted to brain activity.

### CROSS-REFERENCES TO RELATED APPLICATIONS

None

### FEDERALLY FUNDED RESEARCH

None

### BACKGROUND OF THE INVENTION

Since the beginning of civilization, man has searched for peaceful resolution of conflicts. Religious, political, and social philosophers have endorsed peaceful resolution of conflicts among nations as the perfection of world order. However, attempts to achieve this world order through treaties, trade, geographic boundaries, and religious tolerance have failed.

Today's leaders who represent their constituents are the most likely group to promote peaceful resolution of conflicts. As long as they have the following of their constituents, these leaders, whether elected, appointed, or established by other circumstances, are the primary group who can change the present world into a more peaceful world. Pure democratic decisions resolving conflicts do not exist with today's populations, so leaders must govern in republics, monarchies, or dictatorships and make crucial decisions about world order. When conflicts cannot be peacefully negotiated, force, including war, may become the method used to resolve the disputes.

Conflicts are a natural part of human history and peaceful resolutions of conflicts are preferred solutions. Unfortunately, peace negotiations often fail. The cause of these failures are multiple, including poor motivation, lack of creativity, imbalance of power, leadership failure, poor communications, increased aversion to risk, and lack of trust.

### Causes of Conflict

The causes of conflict are numerous, but most depend upon perception of right from wrong. Culture influences many of these perceptions. In each of us, in different frames of reference, there may be a different interpretation of the world around us.[1] In many, these differences may not change behavior, but in some behavior, could change since brain electrical activity is often chaotic, where small changes in perception may result in large changes in the trajectory of thinking.

### The Win-Win Resolution of Conflict

Game theory predicts that conflicts can be resolved with three basic outcomes that include: lose-lose (compromise), lose-win (competitive), and win-win (cooperative). Win-win resolutions are the preferred outcome in which both parties receive more than expected and further negotiation is unlikely to improve the outcomes for either side. Win-win resolution often requires creativity and “out of the box” thinking. Win-win resolution may promote more peaceful resolution of conflicts.

### Deterrence and Conflict

Deterrence has been shown to promote peace through check of the aggressor. The most important factor that determines whether deterrence will be successful is the motivation of the aggressor. Other important factors include the consistency of the responses of the two parties, balance or imbalance of power, and likelihood of a win if an armed conflict ensues. The policy of “firm but flexible” has been adopted to resolve conflict by deterrence.[2] When leaders who possess the machinery to possibly win a war become paranoid, megalomaniacal, and impulsive, they may undertake the risk of war.

### Resolution of Conflict by War

Going to war to settle conflict involves risk. Risk-taking is incorporated in the emotional, cognitive, and physiologic processes in the minds of world leaders. No matter how carefully planned and how abundant are the resources, numerous failed high-risk military interventions in recent world history happen including the Nazi invasion of Russia in WWII (1941), US deployments in Vietnam (1964) and Afghanistan (2001), Israeli invasion of Lebanon (1982), and Iraq invasion of Kuwait (1990).[3]

Deterrence associated with mutually assured destruction (MAD) has probably kept us from nuclear war for decades; however, advances in military technology occur at such a rapid pace that this form of deterrence may not be assured. Furthermore, advances in military technology require large utilizations of resources that usurp funds for the benefit of medical, educational, and environmental programs which could uplift the standards of living for vast populations of societies.

### Conflict Resolution and Negotiation Environment

The environment has effects on the minds of negotiators. Many peace negotiations have been formally conducted in “neutral” environments such as Oslo, Malta, and Geneva. Some peace negotiations have been conducted in symbolic locations such as Gettysburg, or in informal locations such as the ranch of President Ronald Reagan.[4] Whether the negotiations are formal or informal, the meeting of the minds of world leaders is influenced by the environment in which negotiations take place.

### Brain Activity and Behavior

As science learns more about the physiology of the brain, it has become more apparent that the mind is a manifestation of brain activity. Supporting this concept are observations of brain injury associated with personality changes, drug effects on behavior, and electrical stimulation that can change behavior and volition.

Descartes' dualism is probably not a correct theory of the nervous system. Rather, the brain and its manifestation, the mind, is an electrochemical system with stochastic, chaotic, and sometimes periodic activity. In deference to Descartes, electrical and chemical systems are in some ways distinct, and on a macro level, electromagnetic waves are different entities than molecules.

The human brain and the mind associated with it do not exist in a vacuum. The science is quite firm that electrochemical systems do not function without influence from the surroundings. Harnessing the potential benefits of the surrounding environment is something to pursue, because to the best that ordinary citizens know, this improvement could help resolve conflicts and has not been attempted in the past.

Whether electromagnetic waves, electric fields, and magnetic fields have a significant impact on brain functions and concomitant behavior has been contested in the literature. Because the activity of the brain exhibits electrical properties of a chaotic system, it may not take much interference to change the trajectory of brain activity and subsequent behavior.

### Hyperscanning with Partial Synchronization of Brain Activity and Cooperative Resolution of Conflict

Synchronization of oscillating systems was described by Huygens in 1665. At the time, he observed two wall-mounted pendulum clocks that eventually became synchronized over time.[5] Synchronization of natural systems is common, such as the illumination of fireflies, swimming of sperm, cycling of menses, and walking with a partner.[6] In the nervous system, complete synchronization within hemispheres is often associated with seizure activity, but partial synchronization of electrical activity between hemispheres is associated with peaceful states of mind.[7, 8] Also, hyperscanning (concurrent monitoring of brain activity between dyads or among groups) has shown that partial synchronization between dyads and among groups is present during engaged face-to-face communication and cooperative thinking.

At the present time, there are no known drugs that reliably produce partial hemispheric synchronization of the brain, and there are some drugs, such as anticonvulsants, that impede synchronization, which accounts for their anti-seizure activity.[9] Therefore, it seems reasonable to explore electromagnetic interventions directed at the brain that may promote cooperative behavior between dyads and among groups. Such interventions have been shown in models of brain activity to be possible, and these in vitro results may extrapolated to in vivo success.

Although correlating neurophysiology with behavior is fraught with exceptions, many agree that partial intra hemispheric synchronization of brain hemispheric activities may be a hallmark of a peaceful mind. The electroencephalogram (EEG), which measures some of the electrical activity of the brain with scalp electrodes, has been shown to develop partial synchronization of hemispheric brain waves during various forms of meditation and during some auditory stimulation.[8] Partial hemispheric synchronization may occur as the hemispheric brain activity transitions from chaotic activity toward periodic activity. Hyperscanning studies reveal that partial inter brain electrical activity is associated with cooperative thinking.

Partial inter synchronization of brain electrical activity may produce states of mind that are more likely to be associated with cooperative conflict resolutions. There may be many ritual-like behaviors that in the past may have increased partial synchronization between dyads, such as smoking a peace pipe, breaking bread, sharing in a meal, chanting, or praying. There may also be impediments to partial synchronization of brain activity between dyads and groups that include labile emotions, distrust, and language.

In vivo experimentation has shown that stimulation of the scalp with frequency dependent transcutaneous electromagnetic waves at 20 hertz can produce interpersonal synchrony.[10] Also, transcutaneous electrical stimulation at 10 hertz can produce entrained brain oscillations separate from stimulation artifacts.[11]

Extrapolating these findings to brain activity between dyads and among groups may reveal that cooperation can be enhanced when individuals who negotiate are subject to favorable electromagnetic environments that facilitate partial brain synchronization. If this synchronization does not occur, then the leaders should try harder to cooperate, and introduction of electromagnetic energy could be introduced to facilitate cooperation. It is therefore extrapolated that the domicile where negotiations take place should be optimized so that when leaders have a meeting of the minds, it is with partial synchrony of brain activity.[10, 11] Furthermore, it has been shown that entrainment of brain electrical activity is possible through light stimulation.[12]

### Models of Brain Chaotic Activity

Previously it was shown that a chaotic chemical reaction, the Belouvsa-Zhabotinsky (B-Z reaction), could be used to model the electrochemical activity of the brain, and external electromagnetic energy in close proximity to the reaction could change the trajectory of the electrochemical activity.[13] Also, in a similar model, it was shown that electromagnetic waves could interfere with oscillating waves generated by the B-Z reaction.[14] This is not surprising since there are many reports of electromagnetic energy influencing chemical reactions, most well-known being photosynthesis and photoreceptors of rods and cones in the retina. Furthermore, B-Z reactions were shown by other investigators to be influenced by electromagnetic interference.[15, 16]

Previously, it was shown that productions of phosphenes, which are cortical stimulations of the occipital cortex (without retinal stimulation), could be most easily achieved with square wave electrical stimulation, a model of action potentials, rather than sinusoidal or stair step electrical stimulation of the scalp.[17] Also in the same set of experiments, phosphene generation was frequency dependent, occurring between 10 to 35 hertz.[17]

### Language and Conflict

The creator of Esperanto, L. L. Zamenhof, recognize the contribution of a universal second language for peaceful negotiations, and he was nominated for fourteen Nobel Peace Prizes for his work.[18] For many reasons, a universal second language has never been achieved for peace negotiations; however, through the evolution of language, English has become the second language of aviation, the internet, and many aspects of science. English fluency is not easy. Even with the best available translating technology, there is still a difference between direct and indirect communication among leaders during negotiations that may interfere with cooperative inter brain partial synchronization.[19] Face-to-face negotiations with rational and empathetic conversation may foster partial inter brain synchronization and cooperative resolution of conflicts.

This invention extends the in vitro models of simulation of brain activity with Chua's circuits (the simplest model of electrical chaotic activity) to show that partial and complete synchronization of chaotic systems is possible without physical connections between the systems. We have been able to show that two identical Chua's circuits interact with each other through induction, and the circuits became non-chaotic and synchronized. We use this model to explain the partial synchronization of electrical brain activity when dyads are cooperating for resolution of conflict. Monitoring electromagnetic activity of the brains of those involved in negotiations may show the degree of partial synchronization between participants. Such monitoring may direct negotiators to increase their empathy, attention, and trust, as well as improve verbal and gesture communication with their adversaries in order to promote a win-win resolution.

Also, in this invention, using the chaotic Chua's circuit as an electrical model of the brain, it was shown that certain frequencies of electromagnetic energy could change the dynamics of the circuit thereby producing periodic activity and synchrony.

Using the knowledge gained through hyperscanning, it becomes apparent that encouragement of inter brain partial synchronization would be beneficial for cooperative resolution of conflict.

### DESCRIPTION OF THE DRAWINGS

None

### DETAILED DESCRIPTION OF THE INVENTION

At the present time, understanding how distinct areas of the brain produce behavior changes is fraught with many technical difficulties because connectomes are complex, there are a wide a variety of specialized neurons, and structural components such as glial cells and astrocytes are also communicative. Global assessment of activity either through electrical activity or imaging is not perfect but it is the best monitoring that we have at the present time, and electrophysiology is most practical and most temporally precise. Partial intra brain synchronization can occur when hemispheric cerebral electrical activities approach in phase activity, and partial inter brain synchronization can occur when cerebral activities of dyads or groups approach in phase activity.

Hyperscanning studies have shown that cooperative decision-making is associated with partial inter brain synchronization.[20-23] The implications for this finding may be profound for peace negotiations.

In this invention, it was shown that the simplest electrical chaotic circuit, Chua's circuit, could be used to model inter and intra brain activity. The Chua's circuits were constructed according to prior instructions, and then interactions between circuits were investigated modeling inter and intra brain activity. Two identically constructed circuits modeled inter brain activity of dyads, and a single circuit modeled intra brain hemispheric activity.

Furthermore, it was shown that two Chua's circuits could influence each other through induction, without physical connections, changing the activity of both circuits from chaos to synchrony or resonance. Extrapolated to human behavior, it may be possible for two brains to synchronize partially without a physical connection, and such partial inter brain synchronization could enhance cooperative decision-making. Also, it was shown that chaotic activity of Chua's circuit was input frequency dependent. The chaotic system was most sensitive to square wave input, analogous to action potentials, as compared to sine wave input. At a component level, this is not surprising, since imedence of capacitors and inductors are frequency dependent. This finding is in agreement with previous work that cortical stimulation (without retinal stimulation) of the visual system produced phosphenes with square wave scalp stimulation at frequencies below 50 hertz.[17] Also these experiments complement prior experimentation that a chaotic chemical system, the B-Z reaction, can be influenced by external electromagnetic energy.

In this Chua's circuit model, the minimum voltage input square wave frequency to transition from chaotic to synchronized periodic activity was approximately 30 hertz. Therefore, there is vitro experimental data of chaotic systems that can be perturbed by external electromagnetic energy and such perturbations may produce complete or partial synchronization.

### EXPERIMENTAL SECTION

Identical Chua's circuits were constructed on electrical breadboards according to previously described directions, except that the components were soldered in place and powered by an isolated and regulated electrical supply.[24]The chaotic behavior of the systems was confirmed by double scroll imaging on a Tektronix 2445 analog oscilloscope in x-y mode. The chaotic activity of the system was monitored with input of square wave or sine wave via an inductor.

### Experiment #1

Voltage across the inductor of the Chua's circuit was observed in a multi meter while varying frequencies, voltages, and conformations (square and sine wave) were imputed via a second in inductor 1 cm from the inductor in the Chua's circuit. (Table 1)

TABLE 1
Chaos in Chua's circuit-square vs. sine wave

| volts | hertz | conformation | chaos | conformation | chaos |
|---|---|---|---|---|---|
| 1 | 1 | square | + | sine | + |
| 1 | 10 | square | + | sine | + |
| 1 | 20 | square | + | sine | + |
| 1 | 30 | square | − | sine | + |
| 1 | 40 | square | − | sine | + |
| 1 | 50 | square | − | sine | + |
| 1 | 100 | square | − | sine | + |
| 1 | 1,000 | square | − | sine | − |
| 1 | 10,000 | square | − | sine | − |
| 1 | 100,000 | square | − | sine | − |
| 1 | 1,000,000 | square | − | sine | − |

At equal voltage, the response (chaos to synchrony) to square wave input was greater than sine wave input. This is in agreement with magnetic field (B field) calculations. At similar peak to peak voltages with identical solenoids, the square wave magnetic field should be greater than the sine wave magnetic field, because dB/dt is greater with square vs. sine wave. These experiments were conducted in triplicate with similar results and confirmed using an analog oscilloscope. The change in double scroll (chaotic activity) to synchronized activity was qualitatively but not quantitative confirmed that is, driving the system out of chaos with square wave input exceed sine wave input for same voltages and frequencies.

### Experiment #2

The minimum input square wave voltage to drive the system out of chaos at a frequency of 30 hertz was approximately 400 millivolts. These experiments were conducted in triplicate. The results were qualitatively, but not quantitatively confirmed with changes of double scroll (chaotic activity) to synchronized activity that is, increasing voltage changed the activity (Table 2)

TABLE 2
Minimum square wave voltage at
30 hertz vs. chaotic activity in Chua's circuit

| hertz | millivolts | conformation | chaos |
|---|---|---|---|
| 30 | 100 | square | + |
| 30 | 200 | square | + |
| 30 | 300 | square | + |
| 30 | 400 | square | − |
| 30 | 500 | square | − |

### Experiment #3

The effects of distance between the inductors and voltage input at 30 hertz were investigated. At a frequency of 30 hertz with square wave configuration, the distances between inductors to drive the system out of chaos are listed in Table 3.

TABLE 3
Non-chaotic activity with varying distance
between inductors vs. voltage at λ = 30 hertz
with square wave configuration

| volts | Minimum distance (cm) | chaos |
|---|---|---|
| 1 | 1 | − |
| 5 | 2.5 | − |
| 15 | 3.5 | − |
| 20 | 3.5 | − |

### Conclusion from Experiments 1-3

1. External electromagnetic energy, though an inductor, could change the chaotic activity of a Chua's circuit.

2. At equal input voltages with identical inductors, square wave (analogous to action potentials) produces a B field that is more likely to drive this Chua's circuit chaotic system to synchrony than a sine wave conformation.

3. Driving this Chua's circuit model of a chaotic system out of chaos to synchrony was frequency dependent and dependent upon the distance between the inductors.

4. External electromagnetic energy targeted to chaotic biologic systems such as brain activity may be able to change the trajectory of the brain electrical activity.

### Experiment #4

Two identical Chua's circuits, one of which was monitored on an analog oscilloscope, were brought into close proximity such that their inductors were separated by a distance of approximately one centimeter with changing orientations. It was observed that the chaotic systems spontaneously synchronized, implying that coupling was possible through induction without a physical connection between the systems.

### Conclusion from Experiment 4

1. Chaotic electrical systems, which can be models of brain electrical activity, can synchronize through induction, and this could explain in sync activity in biological systems.

2. Hyperscanning with inter brain partial synchrony between dyads in cooperative thinking may be the biological extrapolation of these results.

### Benefits to Society

This invention is not to be considered a panacea for world peace but rather an aid to the negotiation process. At the present time, neuroscience has not determined what specific electromagnetic forces may facilitate inter and intra brain partial synchrony; however hyperscanning monitoring of dyads or groups, if accepted by negotiators, could be useful.

Present estimates of world-wide military expenditures equal approximately two trillion USD. The United States spends more on defense than any other country. If world-wide defense expenditures could be halved, there would exist approximately one trillion USD for health, environmental, and educational programs that would uplift the quality of life on our planet.

If more resolutions of conflict between nations were win-win, nations may be more inclined to trust each other and perhaps there would be less military expenditure. Since win-win resolutions require cooperative rather than competitive or compromise resolutions, partial synchronization of inter brain activity between leaders during peace negotiations may improve outcomes. Such partial synchronization could be monitored, and external electromagnetic stimuli may enhance cooperative activity.

Introducing neuroscience through hyperscanning into conflict negotiations may seem like science fiction, but the in vivo and in vitro scientific data support its use, and the risks of such activity are probably very low. Convincing world leaders to accept monitoring of their brain activity during negotiations will be met with resistance; however, the more we understand the electrophysiology of group decision making, the more likely this addition to resolving conflict will hopefully be accepted. The failure of present day processes to resolve conflicts peacefully among nations should be an incentive to try something new, because peace among nations is so important.

### REFERENCES

1. Goldberg, J. S., REGULATION OF THALAMIC ACTIVITY FOR CONTROL OF AGGRESSION, USPTO, 20190030335A1.
2. Mazarr, M. J., Chan, A., Demus, A., et al, What Deters and Why Exploring Requirements for Effective Deterrence of Interstate Aggression. 2018, Santo Monica, Calif.: RAND Corporation.
3. Vertzberger, Y., Risk taking and decision making: foreign military intervension decisions. 1998, Stanford, Calif.: Stanford University Press. xviii, 519 p.
4. Goldberg, J. S., COHERENT ELECTROMAGNETIC WAVES AID RECONCILIATION, USPTO, 20160302667A1.
5. Oliveira, H. M. and L. V. Melo, Huygens synchronization of two clocks. Sci Rep, 2015. 5: p. 11548.
6. Strogatz, S., SYNC 2015, New York, N.Y.: Hachette Books.
7. Abreu, R., et al., EEG synchronization measures predict epilepsy-related BOLD-fMRI fluctuations better than commonly used univariate metrics. Clin Neurophysiol, 2018. 129(3): p. 618-635.
8. Ferguson, P. C., Psychobiology of Transcendental Meditation-Review. Journal of Altered States of Consciousness, 1975. 2(1): p. 15-36.
9. Nayak, C. S., et al., NREM Sleep and Antiepileptic Medications Modulate Epileptiform Activity by Altering Cortical Synchrony. Clin EEG Neurosci, 2018. 49(6): p. 417-424.
10. Novembre, G., et al., Interpersonal synchrony enhanced through 20 Hz phase-coupled dual brain stimulation. Soc Cogn Affect Neurosci, 2017.
11. Helfrich, R. F., et al., Entrainment of brain oscillations by transcranial alternating current stimulation. Curr Biol, 2014. 24(3): p. 333-9.
12. Notbohm, A., J. Kurths, and C. S. Herrmann, Modification of Brain Oscillations via Rhythmic Light Stimulation Provides Evidence for Entrainment but Not for Superposition of Event-Related Responses. Front Hum Neurosci, 2016. 10: p. 10.
13. Goldberg, J. S., THERMODYNAMIC MODEL OF A NERVOUS SYSTEM, USPTO, 20150379898A1.
14. Goldberg, J. S., METHOD TO MAINTAIN PEACE THROUGH ELECTROMAGNETIC ENERGY TARGETED TO THE BRAIN, USPTO, 20160375220A1.
15. Sevcikova, H. and S. C. Muller, Electric-field-induced front deformation of Belousov-Zhabotinsky waves. Phys Rev E Stat Phys Plasmas Fluids Relat Interdiscip Topics, 1999. 60(1): p. 532-8.
16. Kuze, M., et al., Chemical Wave Propagation in the Belousov-Zhabotinsky Reaction Controlled by Electrical Potential. J Phys Chem A, 2019. 123(23): p. 4853-4857.
17. Goldberg, J. S., METHOD TO OPTIMIZE ELECTRODE PLACEMENT FOR CRANIAL ELECTRICAL STIMULATION, USPTO, 20160129238A1.
18. Grzybowski, A., Ludwik Zamenhof: a major contributor to world culture, on the 150(th) anniversary of his birth. Sury Ophthalmol, 2010. 55(2): p. 183-8.
19. Jiang, J., et al., Neural synchronization during face-to-face communication. J Neurosci, 2012. 32(45): p. 16064-9.
20. Balconi, M. and M. E. Vanutelli, Cooperation and Competition with Hyperscanning Methods: Review and Future Application to Emotion Domain. Front Comput Neurosci, 2017. 11: p. 86.
21. Balconi, M. and M. E. Vanutelli, Brains in Competition: Improved Cognitive Performance and Inter-Brain Coupling by Hyperscanning Paradigm with Functional Near-Infrared Spectroscopy. Front Behav Neurosci, 2017. 11: p. 163.
22. Babiloni, F. and L. Astolfi, Social neuroscience and hyperscanning techniques: past, present and future. Neurosci Biobehav Rev, 2014. 44: p. 76-93.
23. Astolfi, L., et al., Investigating the neural basis of cooperative joint action. An EEG hyperscanning study. Conf Proc IEEE Eng Med Biol Soc, 2014. 2014: p. 4896-9.
24. Gandhi, G., Muthuswamy, B., Roska, T., Chua's Circuit for High School Students. University of California, Berkeley, 2007.

Having described our invention, we claim:

1. A method to improve conflict negotiations comprised of:
a. monitoring the electrophysiology of the brains of those in conflict
b. increasing partial synchronization of the electrical activity of the brains of those in conflict.

2. The method of claim 1, where increasing partial synchronization of the electrical activity of the brains of those in conflict is facilitated by electromagnetic energy targeted to the nervous systems of those in conflict.
